(19) United States
(12) Patent Application Publication
(10) Pub. No.: US 2015/0320820 A1
(43) Pub. Date: Nov. 12, 2015

(54) MICROBIOCIDES FROM PLANT EXTRACTS FOR PROTECTION AGAINST HSV 2 INFECTION

(71) Applicant: Council of Scientific and Industrial Research, New Delhi (IN)

(72) Inventors: Swati Pramod Joshi, Pune (IN); Smita Shrikant Kulkarni, Pune (IN)

(73) Assignee: Council of Scientific and Industrial Research, New Delhi (IN)

(21) Appl. No.: 14/398,313

(22) PCT Filed: May 2, 2013
(86) PCT No.: PCT/IN2013/000293

§ 371 Date: Oct. 31, 2014

(30) Foreign Application Priority Data
May 2, 2012 (IN) 1342/DEL/2012

(51) Int. Cl. A61K 36/704; A61K 36/54; A61K 36/39; A61K 36/72; A61K 36/22; A61K 36/185

(52) U.S. Cl. CPC A61K 36/704; A61K 36/22; A61K 36/185; A61K 36/39; A61K 36/72; A61K 36/54; A61K 2236/39

(57) ABSTRACT

Disclosed herein is a microbicidal composition comprising combination of selected extracts of aerial parts of plants such as Polygonum glabrum, Rhus mysorensis, Terminalia paniculata, Cuscuta reflexa, Terminalia crenulata, Scutia myrtina, and Cassytha filiformis along with pharmaceutically acceptable excipients or carriers, for the treatment of sexually transmitted diseases, particularly HSV-2 type infections.

### TECHNICAL FIELD OF THE INVENTION

The present invention relates to plant derived microbicidal composition against HSV-2 (Herpes simplex virus-2) infection.

Particularly the invention relates to herbal microbicidal composition comprising combination of extracts of aerial parts of plants a) Polygonum glabrum, b) Rhus mysorensis, c) Terminalia paniculata, d) Cuscuta reflexa, e) Terminalia crenulata, f) Scutia myrtina, and g) Cassytha filiformis along with pharmaceutically acceptable excipients or carriers, for the treatment of sexually transmitted diseases, particularly HSV-2 infections.

### BACKGROUND OF THE INVENTION

HSV-2. (which causes genital herpes) is ubiquitous and contagious. Herpes simplex virus (HSV) infection is a common cause of ulcerative mucocutaneous disease in both immune competent and immune compromised individuals. Classically, HSV type 1 (HSV-1) is acquired in childhood and causes orolabial ulcers, whereas HSV type 2 (HSV-2) is transmitted sexually and causes anogenital ulcers. However, both oral infection with HSV-2 and particularly genital infection with HSV-1 are increasingly recognized, likely as a result of oral-genital sexual practices. There are periodic symptomatic reactivation and asymptomatic viral shedding in HSV infection. Infection with HSV is a lifelong condition; the virus becomes permanently latent in the nerve root ganglia corresponding to the site of inoculation (the trigeminal ganglia for orolabial infection and the sacral ganglia for genital infection). HSV induces antibody and cell-mediated immune responses that modulate the severity of recurrent disease, but these are insufficient to eradicate infection.

In the ongoing efforts to reduce the number of HSV fatalities, new approaches are being explored. Secondary metabolites of plants have been found to inhibit HSV and thus biologically active natural products may serve as a good source for microbicide development. Preventive treatments based on plant-derived subunits offer a cost-effective means of combating the growing HSV infections.

Microbicides are pharmacologic agents and chemical substances that are capable of killing or destroying certain microorganisms that commonly cause human infection. They can be effectively used to prevent infection of HIV and other Sexually Transmitted Diseases (STD). They can act as a physical barrier that keeps STD pathogens from reaching the target cells off by defense mechanisms by maintaining an acidic pH or by preventing entry of pathogen and also their replication after it has entered the cell or combination of one or more mechanisms.

Herpes viruses establish lifelong infections and the virus cannot currently be eradicated from the body. Treatment usually involves general-purpose anti-viral drugs that interfere with viral replication, reducing the physical severity of outbreak-associated lesions and lowering the chance of transmission to others.

There are many potential microbicide candidates at various stages in clinical and preclinical trials. In the ongoing effort to stem the tide of HIV fatalities, new approaches are being brought to bear. Plants and other biomaterials are being explored for use as such or as a source of lead molecules for the development of microbicides that would target the virus at its point of entry on the mucosal surfaces of the body (attachment, fusion and entry inhibitors). Biological activity of Polygonum glabrum, Rhus mysorensis, Terminalia paniculata, Cuscuta reflexa, Terminalia crenulata, Scutia myrtina, and Cassytha filiformis is reported in the literature, some of cited herein below:

An ethanol extract of the stems of Polygonum glabrum for anti-inflammatory is disclosed by Bhupinder Singh, in Journal of Ethnopharmacology 19, (3), 1987, pg 255-267. The medicinal uses of the plant Polygonum glabrum (Willd.) (Family: Polygonaceae) such as astringent, diuretic, rubefacient, vermifuge, for the treatment of pneumonia, piles, jaundice, rheumatism, to relieve pain, and for fever is demonstrated by K L. Senthilkumar et al. in International Journal of Pharma and Bio Sciences Vol. 2 Issue 2, 2011. Further In International Journal of Scientific and Research Publications, 2, (9), September 2012 R. N. Maru discloses use of dry root powder Polygonum glabrum Willd. (Polygonaceae) for progenyless and dry stem bark Terminalia crenulata Roth. (Combretaceae) is taken orally to cure heart problems.

Hepatoprotective activity of Rhus mysorensis against carbon tetrachloride induced hepatotoxicity in Albino Rats is reported in International Journal of Pharmaceutical Sciences Review &; September 2010, vol. 4 issue 3, p 46 by Gade.

Cheng H Y et al. in Antiviral Res. 2002 September; 55 (3):447-55 discloses antiviral activity of Casuarinin, a hydrolyzable tannin isolated from the bark of Terminalia arjuna Linn. (Combretaceae), on herpes simplex type 2 (HSV-2) in vitro.

A. R. Srividya in Int J Pahrma Sci Nanotech Vol 4 Issue 4 January-March 2012 discloses cytotoxic, antioxidant and antimicrobial activity of various parts of Polygonum chinensis such as leaf, stem, root and other parts.

Some of the plant, extracts are also useful to inhibit viral infections, wherein inhibition of HIV-1 integrase by galloyl glucoses from Terminalia chebula and flavonol glycoside gallates from Euphorbia pekinensis disclosesd by Mi-Jeong Ahn et al. in Planta Med 2002; 68(5): 457-459.

Arch Virol. 1981; 70(3):215-23 by Awasthi L P discloses aqueous extract of Cuscuta reflexa plants showing highly significant virus inhibiting property.

Article titled “Constituents of Cuscuta reflexa are anti-HIV agents” in Antiviral Chemistry and Chemotherapy (1997) v. 8(1) p. 70-74 by Mahmood, N. reports anti HIV activity of the crude water extracts of Cuscuta reflexa.

GC-MS analysis of phytocomponents in the ethanol extract of Polygonum chinense (whole plant) contained mainly a triterpene compound-squalene and a plasticizer compound-1,2-benzenedicarboxylic acid, mono[2-ethylhexyl]ester having antimicrobial activity, anti-cancer, anti-oxidant, anti-tumor, chemo-preventive is reported in Pharmacognosy Research January 2012, Vol. 4 Issue 1, Ezhilan et al. S. Mythili et al. in Asian Journal of Plant Science and Research, 2011, 1 (1): 77-83 discloses pharmacological activities of Cassytha filiformis such as anti-oxidant, anti-trypanosomal, anti-platelet and vasorelaxant activities, where anti-herpes activity is not demonstrated.

The inventors presented a poster, Abstract for 2010 International Microbicides Conference (M2010) Pittsburgh, Pa., USA Although there are many approaches for preventing sexually transmitted diseases in general and HSV in particular, current methods have not been sufficient to halt the spread of these diseases, particularly among women and people who live in less-developed nations. Therefore, there is a need to develop a cost effective microbicidal composition for protection against sexually transmitted diseases including HSV.

Therefore, the present inventors have come up with novel composition based on plant-derived extracts which offer a cost-effective means for combating the growing sexually transmitted disease pandemic. The invention also supports green chemistry and reduces the side effects due to the use of chemically synthesized drugs.

### SUMMARY OF THE INVENTION

The present invention provides a herbal microbiocidal pharmaceutical composition comprising the combination of extracts of the aerial parts of Polygonum glabrum, Rhus mysorensis, Terminalia paniculata, Cuscuta reflexa, Terminalia crenulata, Scutia myrtina, Cassytha filiformis for the protection against sexually transmitted diseases caused due to pathogens particularly HSV 2, said composition comprising fractions of plant extract as follows:

| Composition | % w/w |
|---|---|
| Polygonum glabrum (Aerial | 7.50-12.00 |
| parts extract no. NCL-2) | |
| Rhus mysurensis (Aerial parts | 7.50-11.50 |
| extract no. NCL-3) | |
| Terminalia paniculata (Fruits | 4.50-6.00 |
| extract no. NCL-4) | |
| Cuscuta reflexa (Aerial parts | 2.00-3.00 |
| extract no. NCL-5) | |
| Terminalia crenulata (Fruits | 2.00-3.00 |
| extract no. NCL-6) | |
| Cassytha filiformis (Aerial parts | 4.50-5.00 |
| extract no. NCL-7) | |
| Scutia myrtima (Aerial parts | 2.00-4.50 |
| extract no. NCL-11) | |
| Pharmaceutical excipients | 65.00-70.00 |

In an aspect, the present invention provides herbal microbicidal composition comprising combination of plant extracts which exhibit microbicidal activity against sexually transmitted disease, HSV-2. The plant extracts are obtained from plants belonging to the families Convolvulaceae, Polygonaceae, Anacardiaceae, Combretaceae, Lauraceae and Rhamnaceae.

### DESCRIPTION OF FIGURES

FIG. 1: FIGS. 1a, 1b and 1 c depicts the HPTLC profiles of compounds NCL-2, 3, 4, 5 and 6 in FIG. 1a, NCL-7 in FIG. 1b and NCL 11 in FIG. 1c.

FIG. 2: FIGS. 2a-2g depicts the HPLC profiles of compounds NCl-2, NCl-3, NCl-4, NCl-5, NCl-6, NCL-7 and NCl-11 respectively.

FIG. 3: Extracts/fractions showing anti-Candida activity. The results are enlisted in Table 5.

FIG. 4: Extracts/fractions showing anti-Gonococci activity. The results are enlisted in Table 6.

FIG. 5: Fractions showing Anti-Ducreyi activity. The results are enlisted in Table 7.

### ABBREVIATIONS

### HSV-1: Herpes Simplex Virus Type-1

### HSV-2: Herpes Simplex Virus Type-2

### HIV: Human Immunodeficiency Virus

CC: Cytotoxic concentration

### IC: Inhibitory Concentrations

### TI: Therapeutic Index

### HPTLC: High Performance Thin Layer Chromatography

### HPLC: High Performance Liquid Chromatography

### DETAILED DESCRIPTION OF THE INVENTION

The invention will now be described in detail in connection with certain preferred and optional embodiments, so that various aspects thereof may be more fully understood and appreciated.

The present invention provides a herbal microbicidal composition comprising extracts of plants for protection against sexually transmitted diseases, particularly HSV-2 infections.

In an embodiment, the present invention provides herbal microbicidal composition comprising combination of plant extracts which exhibit microbicidal activity against sexually transmitted diseases, where the plant extracts are obtained from plants belonging to the families Convolvulaceae, Polygonaceae, Anacardiaceae, Combretaceae, Lauraceae and Rhamnaceae either alone or in combination thereof. The microbicidal activity includes anti-HIV activity against HIV-1, HIV 1 IIIB stain, HIV 1 ada5 strain, HIV 1 UG070 strain, HIV 1 VB59 strain, anti-viral activity against herpes simplex virus-2, antibacterial activity against Neisseria gonorrhea and Haemophilus ducreyi and antifun gal activity against Candida albicans. The sexually transmitted disease particularly includes infections caused due to pathogens such as herpes simplex virus (HSV-1), (HSV-2) and (HIV).

In another embodiment, the present invention provides in vitro HSV screening of plant extracts for potential microbicidal activity, selected on the basis of their ethno-pharmacological and chemo-taxonomical profile.

According to the invention, the plants belong to the families Convolvulaceae, Polygonaceae, Anacardiaceae, Combretaceae, Lauraceae and Rhamnaceae. Particularly, the composition comprises the plant extracts of a) Polygonum glabrum (Polygonaceae), b)Rhus mysorensis (Anacardiaceae), c)Terminalia paniculata (Combretaceae), d) Cuscuta reflexa (Convolvulaceae), e)Terminalia crenulata (Combretaceae), f) Scutia myrtina (Rhamnaceae) and, g) Cassytha filiformis (Lauraceae). The extracts are obtained from various parts of the plants such as aerial parts of the plants by methanol extraction. The methanol extracts are assessed for toxicity and evaluated for anti-viral activity against HSV-2.

According to the embodiment the aerial parts of plants species Polygonum glabrurn, Rhus mysorensis, Terminalia paniculata, Cuscuta reflexa, Terminalia crenulata, Scutia myrtina, Cassytha filiformis include barks, stems, leaves, fruits, flowers, petioles, seeds either alone or combinations thereof.

The invention also provides ethno-pharmacological and chemo-taxonomical based plant selection, which is a good system of identifying newer safer leads that has potential of inhibiting HSV-2 and other sexually transmitted pathogens including HIV-1, which could be moved into microbicide development.

The yield of the extracts of aerial part of said plants with respect to weight of plant part taken as % w/w is: Polygonum glabrum 30-40%, Rhus mysorensis 25-35%, Terminalia paniculata 15-20%, Cuscuta reflexa 5-10%, Terminalia crenulata 5-10%, Scutia myrtina 5-15% and Cassytha filiformis is 15-20%.

In another embodiment, a herbal pharmaceutical composition comprising the combination of extracts of the aerial parts of Polygonum glabrum, Rhus mysorensis, Terminalia paniculata, Cuscuta reflexa, Terminalia crenulata, Scutia myrtina, Cassytha filiformis for the protection against sexually transmitted diseases caused due to pathogens particularly HSV 2 is disclosed.

In another embodiment, the present invention provides a herbal microbicidal composition comprising combination of extracts of aerial parts of plant species a) Polygonum glabrum, b) Rhus mysorensis, c) Terminalia paniculata, d) Cuscuta reflexa, e) Terminalia crenulata, f) Scutia myrtina, and g) Cassytha filiformis or, along with pharmaceutically acceptable excipients or carriers, for the treatment of sexually transmitted diseases particularly herpes simplex virus type 2 (HSV-2) infections in mammals. Generally, the quantity of each active extract present in the range between 2.0% to 12.0% by weight of the composition. Normally, the effective amount of dosage of anti-HSV active component will be in the range of about 0.1 to about 100 mg/kg, more preferably about 1.0 mg to about 50 mg/kg of body weight/day.

The quantity of the plant extracted compound used in instant herbal compositions will vary depending upon the body weight of the patient and the mode of administration and can be of any effective amount to achieve the desired therapeutic effect. The extracted compound of the present invention can also be administered optionally with other therapeutically actives depending on the disease conditions. The instant composition may optionally comprise known antiviral compounds that are effective for treating herpes including: aciclovir (acyclovir), valaciclovir (valacyclovir), famciclovir, and penciclovir.

The herbal composition according to the invention can be formulated in the form of a solid oral formulations such as, tablet, pills, powders, granules, capsules, pellets, beads etc. in the form of topical formulation such as gel, cream, ointment, paste, foam, lotion, transdermal patches, drops, vaginal rings etc. or can be present in the liquid form such as solutions, emulsions, suspensions, syrup, oil etc. or can be used in the form of inhalants or parenteral injection.

According to the invention, the herbal compositions containing the combination of plant extracts may be administered using any effective amount, any form of herbal composition and any route of administration effective for the treatment of sexually transmitted diseases caused due to herpes simplex virus or human immunodeficiency syndrome virus. After formulation, with an appropriate pharmaceutically acceptable carrier in a desired dosage, as known by those of skill in the art, the herbal pharmaceutical compositions of present invention can be administered by any means that delivers the active pharmaceutical ingredient (s) to the site of the body whereby it can exert a therapeutic effect on the patient. The route of administration may comprise systemic, topical, oral and the like.

The excipients or carriers are selected from inert diluents, disintegrants, binders, lubricants, glidants, gelling agents, coatings layer taste-enhancing agents such as saccharin, cyclamate or sugar and other flavourings. They can also comprise suspending excipients or preservatives.

The extracts of the instant plant species are particularly active against sexually transmitted diseases caused due to pathogen such as HSV type 1, HSV type 2 and HIV, preferably HSV-2.

In a specific embodiment, the invention provides methods of inhibiting the growth of herpes simplex virus (HSV-2) which comprises administering an effective amount of said microbicidal composition in association with one or more pharmaceutical excipients or carriers.

Accordingly, the invention provides the use of the said microbicidal composition to inhibit the growth of herpes virus, particularly herpes simplex virus type 2 in mammals.

Accordingly, in an embodiment of the invention, pharmaceutical compositions comprising combination of selected extracts of aerial parts of plant species a) Polygonum glabrum, b) Rhus mysorensis, c) Terminalia paniculata, d) Cuscuta reflexa, e) Terminalia crenulata, f) Scutia myrtina, and g) Cassytha filiformis or its pharmaceutically acceptable salts is disclosed. The range of the extracts is used in the composition in accordance with Table 1.

TABLE 1
The range of the plant extracts used in the composition

| Composition | % w/w |
|---|---|
| Polygonum glabrum (Aerial | 7.50-12.00 |
| parts extract no. NCL-2) | |
| Rhus mysurensis (Aerial parts | 7.50-11.50 |
| extract no. NCL-3) | |
| Terminalia paniculata (Fruits | 4.50-6.00 |
| extract no. NCL-4) | |
| Cuscuta reflexa (Aerial parts | 2.00-3.00 |
| extract no. NCL-5) | |
| Terminalia crenulata (Fruits | 2.00-3.00 |
| extract no. NCL-6) | |
| Cassytha filiformis (Aerial | 4.50-5.00 |
| parts extract no. NCL-7) | |
| Scutia myrtima (Aerial parts | 2.00-4.50 |
| extract no. NCL-11) | |
| Pharmaceutical excipients | 65.00-70.00 |

The following examples, which include preferred embodiments, will serve to illustrate the practice of this invention, it being understood that the particulars shown are by way of examples and for purpose of illustrative discussion of preferred embodiments of the invention only and are not limiting the scope of the invention.

### EXAMPLES

### Example 1

### General Procedure for the Preparation of Herbal Microbicidal Composition

The plant species used in the invention and the geographical origin of the said plants along with other details are as provided in Table 2.

TABLE 2
Details of plant species and their geographical origin

| | | | | Place of | | | |
|---|---|---|---|---|---|---|---|
| | | | | collection | | | |
| | Botanical | | Common | With location | Date of | Voucher | |
| S. No | name | Family | name | coordinates | collection | no. * | Extract % |
| 1. | Polygonum | Polygonaceae | Bihagni | Pune | 19 Sep. 2007 | SPPG1N | 10.98 |
| | glabrum | | | 18°32′10.7″ N | | | |
| | (NCL-2) | | | 73°46′27.9″ E | | | |
| 2. | Rhus | Anacardiaceae | Amboni | Pune | 15 Oct. 2007 | RMNC1L | 11.94 |
| | mysorensis | | | 18°32′7.6″ N | | | |
| | (NCL-3) | | | 73°48′44.9″ E | | | |
| 3. | Terminalia | Combretaceae | Kinjal | Varandha ghat | 24 Dec. 2007 | SPTP2N | 18.82 |
| | paniculata | | | 18°6′17.7″ N | | | |
| | (NCL-4) | | | 73°34′55.2″ E | | | |
| 4. | Cuscuta | Convolvulaceae | Nirmuli, | Lonavala | 23 Oct. 2007 | SPJCR1 | 18.12 |
| | reflexa | | Amarvel | 18°42′54.4″ N | | | |
| | (NCL-5) | | | 73°23′37.9″ E | | | |
| 5. | Terminalia | Combretaceae | Aina | Sinhagad | 16 Oct. 2007 | SPJTC1 | 11.50 |
| | crenulata | | | 18°21′49.1″ N | | | |
| | (NCL-6) | | | 73°45′58.8″ E | | | |
| 6. | Cassytha | Lauraceae | Amarvel | Dapoli | 4 Jan. 2008 | JCTSP9 | 08.54 |
| | filiformis | | | 17°45′17.1″ N | | | |
| | (NCL-7) | | | 73°7′22″E | | | |
| 7. | Scutia | Rhamnaceae | Chimat | Amboli | 23 Apr. 2008 | | 09.88 |
| | myrtina | | | 15°59′40.8″ N | | | |
| | (NCL-0011) | | | 74°74.02′15.8″E | | | |

* Botanical Survey of India, Western Zone, Pune 411 001.

Shade dried and powdered plant materials provided in Table 2, were extracted with methanol (1 kg×3 LX 24 h×3) at room temperature (25 to 30° C.). The individual mixture of each plant species was filtered and concentrated under reduced pressure (50-100 mm Hg) to provide a methanolic extract. The extracts that showed activity were separated into less polar (A), medium polar (B) and polar fractions (C) using column chromatography or solvent separation/partitioning. The procedure was as listed: The methanolic extract, 10 g, was adsorbed on silica gel, 60-120 mesh, 30 g, and chromatographed on silica gel, 100-200 mesh, 200 g, using elution gradient acetone: petroleum ether followed by washing with methanol. Chromatographic fractions were combined into three broad fractions less polar (A), medium polar (B) and polar fractions (C). The methanolic extracts, 20 g, were separated into acetone soluble (A), n-butanol soluble (B) and water soluble (C) fractions

13 wild plant species belonging to eight families Convolvulaceae (3 species), Polygonaceae (2 species), Anacardiaceae (1 species), Combretaceae (2 species), Lauraceae (1 species), Asteraceae (2 species), Zingiberaceae (1 species) and Rhamnaceae (1 species) were selected. Seven of thirteen extracts (CC₈₀: 24-106 μg/ml, IC₈₀:6.88-32 ug/ml, TI: 18-25) and 11 fractions (CC₈₀:8-207 ug/ml, IC₈₀: 7.2-30 ug/ml, TI: 18-201) were found to inhibit cell free HSV2 in the plaque reduction assay.

Plant extracts which exhibited activities were:
1. Polygonum glabrum (Polygonaceae)—Aerial parts (NCL-2)
2. Rhus mysorensis (Anacardiaceae)—Aerial parts (NCL-3)
3. Terminalia paniculata (Combretaceae)—Fruits (NCL-4)
4. Cuscuta reflexa (Convolvulaceae)—Aerial parts (NCL-5)
5. Terminalia crenulata (Combretaceae)—Fruits (NCL-6)
6. Cassytha filiformis (Lauraceae)—Aerial parts (NCL-7)
7. Scutia myrtina (Rhamnaceae)—Aerial parts (NCL-11)

The methanol extracts of the plant parts and their fractions separated as less polar, medium polar and polar fractions using column chromatography or solvent separation/partitioning, were assessed for toxicity and subsequently for inhibition of HSV-2 in Vero cells by plaque reduction assay. These extracts and fractions were also tested for anti-HIV-1 activity and inhibition of other sexually transmitted pathogens like C. albicans, N gonorrhoea and H. ducreyi.

Bio-assay codes for fractions are as follows:
1. NCL-2: NCL-23 (NCL-2A); NCL-24 (NCL-2B); NCL-25 (NCL-2C)
2. NCL-3: NCL-26 (NCL-3A); NCL-27 (NCL-3B); NCL-28 (NCL-3C)
3. NCL-4: NCL-29 (NCL-4A); NCL-30 (NCL-4B); NCL-31(NCL-4C)
4. NCL-5: NCL-32 (NCL-5A); NCL-33(NCL-5B); NCL-34 (NCL-5C)
5. NCL-6: NCL-14 (NCL-6A); NCL-15 (NCL-6B); NCL-16 (NCL-6C)
6. NCL-7: NCL-19 (NCL-7C).
7. NCL-11: NCL-35 (NCL-11A); NCL-36 (NCL-11B); NCL-37 (NCL-11C)

The extracts were characterized by HPLC and HPTLC.

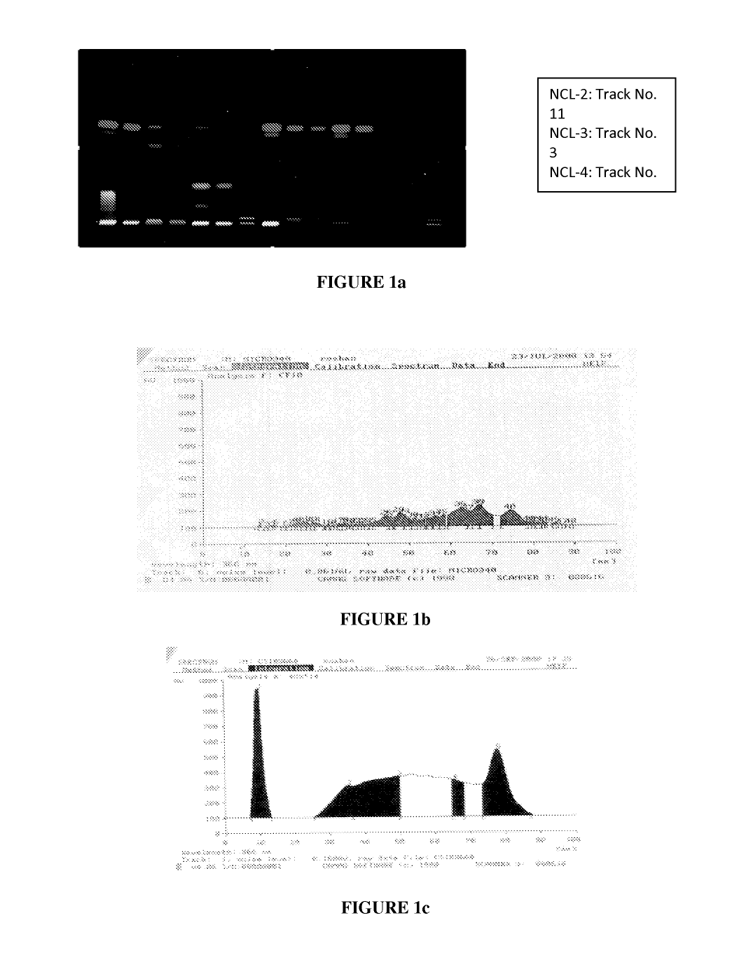

HPTLC Fingerprinting (Refer FIG. 1)

Methanolic extract of plant aerial parts of NCL-2, NCL-3, NCL-4, NCL-5, NCL-6, NCL-7, NCL-11 were dissolved in methanol to get concentration 5 mg/mL. This solution, 10 μL was applied to precoated silica plate GF₂₅₄ (Merck ltd.) using Camag Linomat IV applicator. The plate was developed using developing system ethyl acetate: tolune, 3:7. The plate was scanned at 366 nm using Camag Scanner 3.

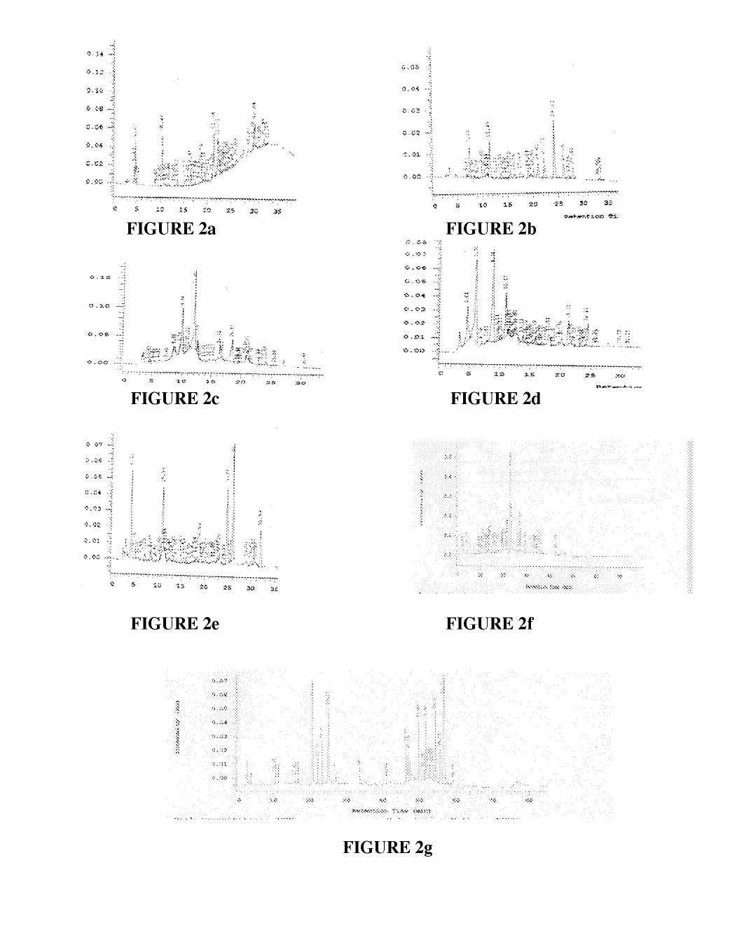

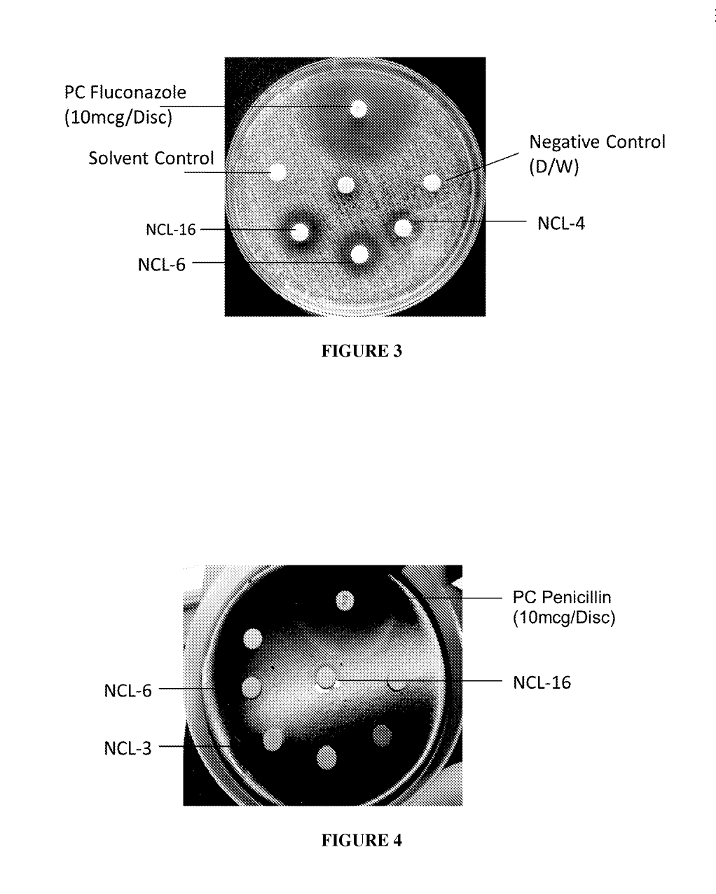

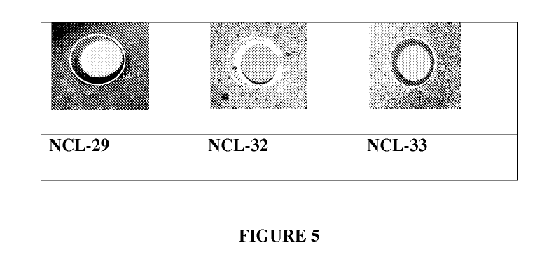

HPLC Fingerprinting (Refer FIG. 2)

### Example 2

Defatted methanolic extract of plant aerial parts of NCL-2, NCL-3, NCL-4, NCL-5, NCL-6, NCL-7, NCL-11 were dissolved in methanol to get concentration 1 mg/mL. This solution, 20 μL, was injected to Merck-Hitachi HPLC instrument equipped with ZORBAX SB-Phenyl (250×4.6 mm) column and mobile phase was acetonitrile 10%: water 90%: formic acid 0.4% (A): acetonitrile (100%) (B) with increasing amount of B (0 to 30% for 40 min., 30%-80% for next 10 min., 80% to 85% for next 10 min., 85% to 100% for next 10 min.). UV detector at 280 nm was used.

These extracts/fractions were effectively used as microbicidal preparation and results are enlisted in Table 3.

TABLE 3
Anti-HSV-2 Plaque assay

| S. | Test | CC₅₀ | CC₈₀ | IC₅₀ | IC₈₀ | |
|---|---|---|---|---|---|---|
| No. | Preparation | μg/ml | μg/ml | μg/ml | μg/ml | TI |
| 1. | NCL-2 | 535 | 329 | 19 | 90 | 29 |
| 2. | NCL-3 | 325 | 164 | 43 | 120 | 8 |
| 3. | NCL-4 | 348 | 67 | 21 | 47.43 | 16 |
| 4. | NCL-5 | 60 | 16 | 9.69 | 28.8 | 6 |
| 5. | NCL-6 | 74 | 24 | 2.98 | 6.88 | 25 |
| 6. | NCL-7 | 333 | 106 | 14 | 32 | 29 |
| 7. | NCL-11 | 147 | 56 | 8 | 30 | 18 |
| 8. | NCL-15 | 397 | 81 | 18 | 60 | 22 |
| 9. | NCL-16 | 461 | 98 | 11.5 | 26 | 40 |
| 10. | NCL-19 | 184 | 94 | 50 | 117 | 4 |
| 11. | NCL-23 | 332 | 53 | 9 | 30 | 38 |
| 12. | NCL-24 | 410 | 132 | 30 | 63 | 13 |
| 13. | NCL-25 | 80 | 8 | 4.5 | 21.8 | 18 |
| 14. | NCL-26 | 493 | 207 | 13 | 38 | 37 |
| 15. | NCL-27 | 140 | 42 | 4 | 20.4 | 31 |
| 16. | NCL-28 | 11125 | 387 | 276 | 1458 | 59 |
| 17. | NCL-29 | 128 | 35 | 10 | 29 | 13 |
| 18. | NCL-30 | 181 | 21 | 9 | 29.04 | 20 |
| 19. | NCL-31 | 41 | 10 | 9 | 28.82 | 4 |
| 20. | NCL-32 | 276 | 119 | 2.56 | 7.2 | 108 |
| 21. | NCL-33 | 58 | 6 | 4.54 | 24 | 13 |
| 22. | NCL-35 | 622 | 82 | 3.1 | 10.33 | 201 |
| 23. | NCL-36 | 294 | 183 | 9 | 21.7 | 34 |
| 24. | NCL-37 | 1602 | 780 | 69.4 | 206 | 23 |
| 25. | Acyclovir | 806 | 378 | 5.6 | 2.81 | 151 |
| | (Positive | | | | | |
| | Control) | | | | | |
| 26. | DMSO solvent | 111 | 35 | 61 | 150.81 | 2 |

According to Table 3, the extracts of plant species selected from the group consisting of Polygonum glabrum, Rhus mysorensis, Terminalia paniculata, Cuscuta reflexa, Terminalia crenulata, Cassytha filiformis, Scutia myrtina, were studied for cytotoxicity in Vero cells. (CC₅₀ in the range, of 60 to 540 μg/ml; CC₈₀ in the range of 20 to 340 μg/ml). The said plant extracts exhibited the anti-HSV activities with IC₅₀ in the range of 2 to 50 μg/ml; IC₈₀ in the range of 6 to 130 μg/ml; and TI in the range of 6 to 29.

### Example 3

Pharmaceutical powder comprising composition as given in Table 4 has been prepared by following procedure:

Dissolve mannitol in water, add flavor to it.

Evaporate water to adsorb color and flavor on mannitol.

Mix active ingredient and Magnesium stearate and fill in pouch or bottle.

Mode of administration: Disperse the powder in water/juice.

TABLE 4
Composition of pharmaceutical powder

| Composition | % w/w |
|---|---|
| Polygonum glabrum | 7.50 |
| Rhus mysurensis | 7.50 |
| Terminalia paniculata | 4.50 |
| Cuscuta reflexa | 2.00 |
| Terminalia crenulata | 2.00 |
| Cassytha filiformis | 4.50 |
| Scutia myrtima | 2.00 |
| Raspberry Flavor | 1.0 |
| Magnesium stearate | 2.0 |
| Manitol | q.s. to 100% |

Pharmaceutical gel preparation procedure: Gel formulation was prepared by dispersing 3% w/w Hydroxypropylmethylcellulose, (HPMC) in water by continuous stirring for a period of 2 h. Extracts as given in Table 5 were dissolved in ethanol and the solution was added gently to HPMC, under continuous stirring. The mixture was stirred gently until homogeneous gel was formed and filled in appropriate container such as bottle or tube.

Mode of administration: Apply gently on affected area.

TABLE 5
Composition of pharmaceutical gel preparation

| Composition | % w/w |
|---|---|
| Polygonum glabrum | 0.75 |
| Rhus mysurensis | 0.75 |
| Terminalia paniculata | 0.45 |
| Cuscuta reflexa | 0.20 |
| Terminalia crenulata | 0.20 |
| Cassytha filiformis | 0.45 |
| Scutia myrtima | 0.20 |
| Ethanol | 10.0 |
| Hydroxypropylmethylcellulose (HPMC) | 3.00 |
| Water | q,s. to 100% |

### Example 4

### Activity Against Candida and Gonococci

### Microbial Strains

The standard strains of Neisseria gonorrhoeae, Haemophilus duceryi and Candida albicans were obtained from the American Type culture Collection. The laboratory strains isolated from the clinical specimens were obtained from the Microbiology Department, National AIDS Research Institute, Pune, India.

Neisseria gonorrhoeae

Two penicillin sensitive (ATCC-49226, NARI-1511) and penicillin resistant (ATCC-49926, NARI-1562) strains of N. gonorrhoeae were used for testing the activity. All the strains were cultured on the modified Thayer Martin medium and incubated at 37° C. for 48 hrs in a candle jar with 45% of moisture. The isolates were further identified by colony characteristics, Gram stain and Rapid Carbohydrate Utilization Test as described earlier, and stored at −70° C. in nutrient broth containing 20% glycerol.

Haemophilus duceryi

A tetracycline sensitive strain of H. duceryi (ATCC-39921) was cultured on Chanchroid agar (GC agar supplemented with 2% haemoglobin, 5% FBS and 1% isovitalex and incubated at 33° C. for 72 hrs. in a candle jar with 45% moisture. The strain was identified by colony characteristics, Gram stain, growth characteristics (requirement for X and V factors) and Tween 80 hydrolysis as described earlier, and stored at −70° C. in brain heart infusion broth containing 20% glycerol.

Candida albicans

Two fluconazole sensitive (ATCC-90028, NARI-71) and one fluconazole resistant (NARI-68) C. albicans strains were used for testing the activity. All strains were cultured on Sabouraud's dextrose agar (SDA). The strains were first identified by the Gram stain and then by the colour of colonies on Hichrome agar (HiMedia Laboratories, India). The strains were confirmed by using germ tube method and API biochemical test (Biomeriux, SA) as described earlier (Sheppard et. al. 2008). The isolates were stored in Sabouraud's dextrose broth containing 20% glycerol at −70° C.

### Susceptibility Testing

### Preparation of the Inoculums

The inoculum of each strain was prepared following the CLSI guidelines for bacteria and yeasts. The 0.5 McFarland standard inoculums (approx. 1.5×10⁸ CFU/ml) of N. gonorrhoeae, H. duceryi and C. albicans were prepared from freshly grown cultures, in sterile 0.85% normal saline and mixed thoroughly.

### Preparation of the Discs of Plant Extracts/Fractions

For each experiment, 6 mm sterile filter paper discs (HiMedia Laboratories, India) were freshly prepared by dispensing 20 μl of the plant extract/fraction (10 mg/ml). Similarly, solvent control and negative control discs (distilled water) were prepared and used as controls while testing the extracts/fractions.

1. Evaluation of the Anti-Microbial Activity by Disc Diffusion Method

The primary screening of plant extracts for activity against the three microorganisms included in the study was carried out by Kirby Baur agar disc diffusion method as described earlier. Each experiment was carried out in duplicate and repeated three times. The discs (extract/fraction and controls) were placed on the plates previously inoculated with the respective bacterial/fungal culture, incubated as mentioned above and the zone of inhibition was recorded. The fluconazole (10 mcg), penicillin (10 μg) and tetracycline (10 μg) discs (HiMedia Laboratories, India) were used as positive controls for comparing activity against N. gonorrhoeae, H. duceryi and C. albicans respectively (Talwar et. al. 2000, Rahiem et. al. 2005). The minimum inhibitory and minimum cidal concentrations of the extracts showing inhibition were determined using following methods.

2. Determination of the Minimum Inhibitory and/Minimum Cidal Concentrations

Neisseria gonorrhoeae

The minimum cidal concentration (MCC) of the extracts showing anti-gonococcal activity was further determined using following method. The extracts were diluted as mentioned earlier (78 to 10000 μg/ml). To 90 μl of the diluted extract, 10 μl of the inoculum (1:50 dilution of 0.5 McFarland inoculum) was added and the plates were incubated at 37° C. for 30 min in presence of 5% CO₂. The extract/fraction/positive control and bacterial inoculum mixture (24 μl) was added on the pre-labeled GC agar plate and the plates were incubated for 24 hrs at 37° C. in candle jar encompassing 5% CO₂ atmosphere. After incubation, the number of colonies were counted and compared with the number of colonies in absence of drug or extract/fraction.

Haemophilus ducreyi: Zone of inhibition of active fractions (in mm) is given in Table 7.

Candida albicans

The minimum inhibitory concentration (MIC) and the minimum cidal concentration (MCC) of the extracts/fractions showing anti-candida activity was further determined using micro-broth dilution method. For determining MIC, 100 μl of the serial, two-fold dilutions (78 to 10000 μg/ml) of each plant extracts/fractions were prepared in RPMI 1640, Sigma, USA. Subsequently, 100 μl of the inoculum (1×10³ CFU/ml) was added to each well and the plates were incubated at 35° C. for 48 hrs. After incubation, the turbidity was measured visually. Fluconazole as controls were included with every set of experiment.

For determining MCC, 10 μl of suspension from each well of MIC plate was, inoculated on the SDA plate in duplicate. The plates were incubated for 24 hrs at 37° C. After incubation, the number of colonies were counted and compared with the number of colonies in absence of drug or extract/fraction.

### Anti-Candida Testing

TABLE 6
Extracts/sub fractions showing anti-Candida activity by agar disc diffussion

| | | Test | Candida albicans | | |
|---|---|---|---|---|---|
| | Institution | Extract | ATCC | Lab Isolates | |
| S. No. | Code | Conc. | 90028 (S) | 71 (S) | 68 (R) |
| 1 | NCL-4 | 100 mg/ml | 11 mm (±2.2)* | 10 mm (±0.09) | 10 mm (±2) |
| | | 5 mg/ml | — | — | — |
| 2 | NCL-6 | 10 mg/ml | 15 mm (±1.1) | 10 mm (±2.9) | 10 mm (±1.7) |
| | | 5 mg/ml | 11 mm (±1.9) | 8 mm (±1.4) | 10 mm (±0.3) |
| | | 2.5 mg/ml | 8 mm (±1.5) | — | — |
| 3 | NCL-16 | 10 mg/ml | 13 mm (±1.1) | 11 mm (±0.8) | |

### Anti-Gonococci Testing

| | | Neisseria gonorrohoeae | | | |
|---|---|---|---|---|---|
| | | ATCC | | Lab Isolates | |
| S. No. | Product No | 49226 (S) | 49226 (R) | 1511 (S) | 1562 (R) |
| 1. | NCL-3 | — | — | 7 mm (±0.2) | — |
| 2. | NCL-4 | — | — | — | — |
| 3. | NCL-5 | — | 7 mm (±0.5) | 7 mm (±0.7) | — |
| 4. | NCL-6 | 10 mm (±0.6) | 7 mm (±0.6) | 8 mm (±0.9) | 8 mm (±0.5) |
| 5. | NCL-16 | 10 mm (±0.6) | 10 mm (±0.5) | 11 mm (±0.9) | 10 mm (±0.4) |
| 6. | NCL-29 | 10 mm (±0.6) | 10 mm (±0.6) | — | 10 mm (±0.6) |
| 7. | NCL-30 | 10 mm (±0.5) | 10 mm (±0.6) | — | 10 mm (±0.4) |

TABLE 8
Anti-Ducreyi activity

| | | Haemophilus duceryi ATCC 33921 | |
|---|---|---|---|
| S. No. | Product | (Tetracycline Sensitive) | |
| 1. | NCL-29 | 8 mm | (±0.7) |
| 2. | NCL-32 | 8 mm | (±0.3) |
| 3. | NCL-33 | 8 mm | (±0.5) |
| 4. | penicillin (10 μg) | 36 | (±0.6) |

| | | |
|---|---|---|
| 5. | Solvent Control | — |

1. A herbal microbicidal composition, the composition comprising a combination of selected extracts of aerial parts of plants comprising Polygonum glabrum 7.50-12.00%, Rhus mysurensis 7.50-11.50%, Terminalia paniculata 4.50-6.00-%, Cuscuta reflexa 4.50-6.00-%, Terminalia crenulata 2.00-3.00%, Scutia myrtima 2.00-4.50% and Cassytha filiformis 4.50-5.00-% by weight of the composition along with pharmaceutically acceptable excipients or carriers, for the treatment of sexually transmitted diseases.

2. The composition according to claim 1, wherein the aerial parts are comprised of barks, stems, leaves, fruits, flowers, petioles, seeds either alone or combinations thereof.

3. The composition according to claim 1, wherein the selected extract of plant material used are methanol extracts of the plant parts and their fractions being separated as less polar, medium polar and polar fractions using column chromatography or solvent separation/partitioning.

4. The composition according to claim 1, wherein the sexually transmitted diseases comprise infections due to pathogens selected from group consisting of herpes simplex virus type-1 (HSV-1), herpes simplex virus type 2 (HSV-2).

5. The composition according to claim 1, comprising an additional active ingredient such as herein described, together with pharmaceutically acceptable excipients and/or vehicles wherein excipients or carriers are selected from inert diluents, disintegrants, binders, lubricants, glidants, gelling agents, coatings layer taste-enhancing agents such as saccharin, cyclamate or sugar.

6. The composition according to claim 1, wherein the composition obtained is being formulated in the form of solid oral formulations such as tablets, pills, powders, granules, capsules, pellets, or beads, in the form of a topical formulation such as gel, cream, ointment, paste, foam, lotion, transdermal patches, drops, or vaginal rings.

7. The composition according to claim 1, wherein the selected plant extracts used exhibited the anti-HSV activities with IC₅₀ in the range of 2 to 50 μg/ml; IC₈₀ in the range of 6 to 130 μg/ml; and TI in the range of 6 to 29.

8. A method of treating or inhibiting herpes simplex virus infections with the composition as claimed in claim 1 comprising administering the composition with one additional active compound together as herein described with pharmaceutically acceptable excipients and/or vehicles.

9. Use of microbicidal composition according to claim 1 for the preparation of a medicament useful for treating or inhibiting sexually transmitted disease in mammals, wherein the sexually transmitted disease is caused due to pathogens, particularly herpes simplex virus.

10. Use of microbicidal composition according to claim 1, wherein, the sexually transmitted disease is caused due to Herpes Simplex Virus type 2.
